(19) United States
(12) Patent Application Publication
(10) Pub. No.: US 2001/0039006 A1
(43) Pub. Date: Nov. 8, 2001

(54) Toxicity typing using embryoid bodies

(76) Inventor: H. Ralph Snodgrass, San Mateo, CA (US)

Correspondence Address: Gladys H. Monroy, Morrison & Foerster LLP, 755 Page Mill Road, Palo Alto, CA, 94304-1018, US

(21) Appl. No.: 09/864,621

(22) Filed: May 23, 2001

Related U.S. Application Data
(62) Division of application No. 09/457,931, filed on Dec. 8, 1999.
(60) Provisional application No. 60/111,640, filed on Dec. 9, 1998.

(51) Int. Cl. C12Q 1/00; C12N 5/08

(52) U.S. Cl. 435/4; 435/366

(57) ABSTRACT

This invention provides methods and systems for identifying and typing toxicity of chemical compositions, as well as for screening new compositions for toxicity. The invention involves detecting alterations in gene or protein expression and hence establishing molecular profiles in isolated mammalian embryoid bodies contacted with various chemical compositions of known and unknown toxicities, and correlating the molecular profiles with toxicities of the chemical compositions.

### CROSS-REFERENCE TO RELATED APPLICATIONS

This application is a divisional application of application Ser. No. 09/457,931, filed Dec. 8, 1999, which claims priority under 35 USC §119(e) to U.S. Provisional Application Ser. No. 60/111,640, filed Dec. 9, 1998, the entire contents of which are incorporated herein by reference.

### TECHNICAL FIELD

This invention provides methods for identifying and characterizing toxic compounds as well as for screening new compounds for toxic effects.

### BACKGROUND ART

Some 55,000 chemicals are currently produced or used in the United States every year. Relatively few of these compounds have undergone comprehensive testing for acute or chronic toxicities. One estimate is that less than 1 percent of commercial chemicals have undergone a complete health hazard assessment. Faster and less expensive means of testing the toxicity of these compounds would be desirable. It would be particularly useful if such means were also amenable to high throughput use.

In addition to industrial and household chemicals, a number of chemical compositions are developed each year for use as pharmaceuticals. Rules regarding the testing of potential pharmaceuticals are promulgated by the Food and Drug Administration (“FDA”), which currently requires comprehensive testing of toxicity, mutagenicity, and other effects in at least two species, only one of which can be murine, before a drug candidate can be entered into human clinical trials. Preclinical toxicity testing alone costs some hundreds of thousands of dollars.

In 1997, the pharmaceutical industry was estimated to have spent over $4.5 billion on screening assays and testing to determine toxicity. Despite this huge investment, almost one third of all prospective human therapeutics fail in the first phase of human clinical trials because of unexpected toxicity. It is clear that currently available toxicological screening assays do not detect all toxicities associated with human therapy. Better means of screening potential therapeutics for potential toxicity would reduce the cost and uncertainty of developing new therapeutics and, by reducing uncertainty, would encourage the private sector to commit additional resources to drug development.

Currently available alternatives to traditional “single-reporter” cell lines and animal toxicity testing do not fully meet these needs. For example, Farr, U.S. Pat. No. 5,811,231, provides methods of identifying and characterizing toxic compounds by choosing selected stress promoters to and determining the level of the transcription of genes linked to these promoters in cells of various cell lines. This method therefore depends on the degree to which both the promoter and the cell lines are representative of the effect of the potentially toxic agent on the organism of interest.

The use of hybridization arrays of oligonucleotides provides another route for determining the potential toxicity of chemical compositions. Exposing cells of a culture to a chemical composition and then comparing the expression pattern of the exposed cells to that of cells exposed to other chemical agents permits one to detect patterns of expression similar to that of the test compound, and thus to predict that the toxicities of the chemical compositions will be similar. See, e.g., Service, R., Science 282:396-399 (1998). These methods suffer from the fact that individual cell lines may not be fully representative of the complex biology of an intact organism. Moreover, even repeating the tests in multiple cell lines does not reproduce or account for the complex interactions among cells and tissues that occurs in an organism.

What is needed in the art is a method of systematically testing chemical compositions for potential toxicity in a milieu in which cells interact with cells of other types. What is further needed is a means of doing so which is relevant to the effect of the composition on whole organisms, without the cost, time, and ethical ramification of animal and human testing. The present invention addresses these and other needs.

### DISCLOSURE OF THE INVENTION

This invention provides novel methods for assessing the toxicity of chemical compositions. In one group of embodiments, the invention is directed to methods of creating a molecular profile of a chemical composition, comprising the steps of a) contacting an isolated mammalian embryoid body (EB) with the chemical composition; and b) recording alterations in gene expression or protein expression in the mammalian embryoid body in response to the chemical composition to create a molecular profile of the chemical composition.

The invention further embodies methods of compiling a library of molecular profiles of chemical compositions having predetermined toxicities, comprising the steps of a) contacting an isolated mammalian embryoid body with a chemical composition having predetermined toxicities; b) recording alterations in gene expression or protein expression in the mammalian embryoid body in response to the chemical composition to create a molecular profile of the chemical composition; and c) compiling a library of molecular profiles by repeating steps a) and b) with at least two chemical compositions having predetermined toxicities.

Another embodiment of the present invention provides methods for typing toxicity of a test chemical composition by comparing its molecular profile in EB cells with that of an identified chemical composition with predetermined toxicity. In one aspect, the test chemical composition can be the same as the chemical composition having predetermined toxicities. For example, the test chemical is identified through this testing as exhibiting the identical molecular profile as the known chemical composition.

The invention further encompasses systemic methods for typing the toxicity of a test chemical composition by making the profile comparison with a library comprising profiles of multiple chemical compositions with predetermined toxicities. Preferably, the chemical compositions comprised in a library exert similar toxicities in terms of types and target tissues or organs. The library can be in the form of a database. A database may comprise more than one library for chemical compositions of different toxicity categories.

In one aspect of the present invention, the toxicity of a test chemical composition can be ranked according to a comparison of its molecular profile in EB cells to those of chemical compositions with predetermined toxicities.

Embryoid bodies in the present invention can be of human or non-human mammals, including those of murine species, as well as canine, feline, porcine, bovine, caprine, equine, and sheep species.

The alterations in levels of gene or protein expression can be detected by use of a label selected from any of the following: fluorescent, calorimetric, radioactive, enzyme, enzyme substrate, nucleoside analog, magnetic, glass, or latex bead, colloidal gold, and electronic transponder. The alterations can also be detected by mass spectrometry. The chemical composition can be known (for example, a potential new drug) or unknown (for example, a sample of an unknown chemical found dumped near a roadside and of unknown toxicity).

Further, the chemical compositions can be therapeutic agents (or potential therapeutic agents), of agents of known toxicities, such as neurotoxins, hepatic toxins, toxins of hematopoietic cells, myotoxins, carcinogens, teratogens, or toxins to one or more reproductive organs. The chemical compositions can further be agricultural chemicals, such as pesticides, fungicides, nematicides, and fertilizers, cosmetics, including so-called “cosmeceuticals,” industrial wastes or by-products, or environmental contaminants. They can also be animal therapeutics or potential animal therapeutics.

The invention further includes integrated systems for comparing the molecular profile of a chemical composition to a library of molecular profiles of chemical compositions, comprising an array reader adapted to read the pattern of labels on an array, operably linked to a computer comprising a data file having a plurality of gene expression or protein expression profiles of mammalian embryoid bodies contacted with known or unknown chemical compositions.

The invention also includes integrated systems for correlating the molecular profile and toxicity of a chemical composition comprising an array reader adapted to read the pattern of labels on an array, operably linked to a digital computer comprising a database file having a plurality of molecular profiles of chemical compositions with predetermined toxicities and a program suitable for molecular profile-toxicity correlation. The integrated systems of the invention can be capable of reading more than 500 labels in an hour, and further can be opeably linked to an optical detector for reading the pattern of labels on an array.

### BRIEF DESCRIPTION OF THE DRAWINGS

FIG. 1 depicts differences in expression of nuclear proteins between embryoid bodies exposed to one of two drugs, and control embryoid bodies.

FIG. 1A is a half-tone reproduction of a readout from the mass spectrometer. The top band is the mass spectrum for control embryoid bodies, which were grown in the absence of either of the test chemical compositions. The middle band is the mass spectrum for the embryoid bodies grown in the presence of added troglitazone, and the bottom band of FIG. 1A shows the mass spectrum of nuclear proteins expressed by embryoid bodies exposed to erythromycin estolate.

FIGS. 1B and 1C are bar graphs that represent computational subtractions of identical proteins between the respective test embryoid bodies and the control embryoid bodies to indicate only those proteins which are significantly different in expression between the test and the control embryoid bodies. Each bar represents a single protein and the height of the bar represents the amount of protein expressed by the embryoid bodies exposed to the test composition compared to the amount expressed by embryoid bodies not exposed to the chemical composition.

FIG. 1B: protein expression of test embryoid bodies contacted with troglitazone compared to protein expression of controls.

FIG. 1C: protein expression of test embryoid bodies contacted with erythromycin estolate compared to protein expression of controls.

FIG. 2 is a bar graph showing expression of small nuclear proteins detected by mass spectrometry. X-axis: mass of protein detected. Y-axis: amount of protein detected, in relative units.

FIG. 2A: Protein expression of control embryoid bodies not exposed to the chemical composition.

FIG. 2B: Protein expression of embryoid bodies exposed to troglitazone.

FIG. 2C: Protein expression of embryoid bodies exposed to erythromycin estolate. Bold lines indicate proteins expressed in different amounts between embryoid bodies exposed to troglitazone and those exposed to erythromycin estolate.

FIG. 3 is a bar graph showing expression of small cytoplasmic proteins detected by mass spectrometry. X-axis: mass of protein detected. Y-axis: amount of protein detected, in relative units.

FIG. 3A: Protein expression of control embryoid bodies not exposed to the chemical composition.

FIG. 3B: Protein expression of embryoid bodies exposed to troglitazone.

FIG. 3C: Protein expression of embryoid bodies exposed to erythromycin estolate. Bold lines indicate proteins expressed in different amounts between embryoid bodies exposed to troglitazone and those exposed to erythromycin estolate.

FIG. 4 is a bar graph showing expression of large nuclear proteins detected by mass spectrometry. X-axis: mass of protein detected. Y-axis: amount of protein detected, in relative units.

FIG. 4A: Protein expression of control embryoid bodies not exposed to the chemical composition.

FIG. 4B: Protein expression of embryoid bodies exposed to troglitazone.

FIG. 4C: Protein expression of embryoid bodies exposed to erythromycin estolate. Bold lines indicate proteins expressed in different amounts between embryoid bodies exposed to troglitazone and those exposed to erythromycin estolate.

### MODE(S) FOR CARRYING OUT THE INVENTION

A. Definitions

As used herein, “embryoid body”, “EB” or “EB cells” typically refers to a morphological structure comprised of a population of cells, the majority of which are derived from embryonic stem (“ES”) cells that have undergone differentiation. Under culture conditions suitable for EB formation (e.g., the removal of Leukemia inhibitory factor or other, similar blocking factors), ES cells proliferate and form small mass of cells that begin to differentiate. In the first phase of differentiation, usually corresponding to about days 1-4 of differentiation for humans, the small mass of cells forms a layer of endodermal cells on the outer layer, and is considered a “simple embryoid body.” In the second phase, usually corresponding to about days 3-20 post-differentiation for humans, “complex embryoid bodies” are formed, which are characterized by extensive differentiation of ectodermal and mesodermal cells and derivative tissues. As used herein, the term “embryoid body” or “EB” encompasses both simple and complex embryoid bodies unless otherwise required by context. The determination of when embryoid bodies have formed in a culture of ES cells is routinely made by persons of skill in the art by, for example, visual inspection of the morphology. Floating masses of about 20 cells or more are considered to be embryoid bodies. See. e.g., Schmitt, R., et al. (1991) Genes Dev. 5:728-740; Doetschman, T. C., et al. (1985) J. Embryol. Exp. Morph. 87:27-45. It is also understood that the term “embryoid body,” “EB,” or “EB cells” as used herein encompasses a population of cells, the majority of which being pluripotent cells capable of developing into different cellular lineages when cultured under appropriate conditions. As used herein, the term also refers to equivalent structures derived from primordial germ cells, which are primitive cells extracted from embryonic gonadal regions. See, e.g., Shamblott, et al. (1998) Proc Natl Acad Sci (USA) 95:13726-13731. Primordial germ cells, sometimes also referred to in the art as ES cells or embryonic germ cells, when treated with appropriate factors form pluripotent ES cells from which embryoid bodies can be derived. See, e.g., Hogan, U.S. Pat. No. 5,670,372; Shamblott, et al., supra.

“Toxicity,” as used herein, means any adverse effect of a chemical on a living organism or portion thereof. The toxicity can be to individual cells, to a tissue, to an organ, or to an organ system. A measurement of toxicity is therefore integral to determining the potential effects of the chemical on human or animal health, including the significance of chemical exposures in the environment. Every chemical, and every drug, has an adverse effect at some concentration; accordingly, the question is in part whether a drug or chemical poses a sufficiently low risk to be marketed for a stated purpose, or, with respect to an environmental contaminant, whether the risk posed by its presence in the environment requires special precautions to prevent its release, or quarantining or remediation once it is released. See, e.g., Klaassen, et al., eds., Casarett and Doull's Toxicology: The Basic Science of Poisons, McGraw-Hill (New York, N.Y., 5^{th} Ed. 1996). As used herein, a chemical composition with “predetermined toxicities” means that the type of toxicities and/or certain pharmacodynamic properties of the chemical composition have been determined. For example, a chemical composition may be known to induce liver toxicity. Furthermore, the severity of liver toxicity caused by the chemical may be quantitatively measured by the amount or concentration of the chemical in contact with the liver tissues.

“Alteration in gene or protein expression” according to the present invention means a change in the expression level of one or more genes or proteins compared to the gene or protein expression level of an embryoid body which has been exposed only to normal tissue culture medium and normal culturing conditions. Depending on the context, the phrase can mean an alteration in the expression of a single protein or gene, as when an embryoid body exposed to a chemical agent expresses a protein not expressed by a control embryoid body, or it can mean the overall pattern of protein expression of an embryoid body (or group of embryoid bodies).

“Chemical composition,” “chemical,” “composition,” and “agent,” as used herein, are generally synonymous and refer to a compound of interest. The chemical can be, for example, one being considered as a potential therapeutic, an agricultural chemical, an environmental contaminant, or an unknown substance found at a crime scene, at a waste disposal site, or dumped at the side of a road.

As used herein, “molecular profile” or “profile” of a chemical composition refers to a pattern of alterations in gene or protein expression, or both, in an embryoid body contacted by the chemical composition compared to a like embryoid body in contact only with culture medium.

As used herein, “database” refers to an ordered system for recording information correlating information about the toxicity, the biological effects, or both, of a chemical agent to the alterations in the pattern of gene or protein expression, or both, in an embryoid body contacted by a chemical composition compared to a like embryoid body in contact only with culture medium.

A “library,” as used herein, refers to a compilation of molecular profiles of at least two chemical compositions, permitting a comparison of the alterations in gene or protein expression, or both, in an embryoid body contacted by a chemical composition to the profiles of such expression(s) caused by other chemical compositions. “Array” means an ordered placement or arrangement. Most commonly, it is used herein to refer to an ordered placement of oligonucleotides (including cDNAs and genomic DNA) or of ligands placed on a chip or other surface used to capture complementary oligonucleotides (including cDNAs and genomic DNA) or substrates for the ligand. Since the oligonucleotide or ligand at each position in the arrangement is known, the sequence (of a nucleic acid) or a physical property (of a protein) can be determined by the position to which the nucleic acid or substrate binds to the array.

“Operably linked” means that two or more elements are connected in a way that permits an event occurring in one element (such as a reading by an optical reader) to be transmitted to and acted upon by a second element (such as a calculation by a computer concerning data from an optical reader).

### B. General Description

The invention provides methods of assessing toxicity of chemical compositions on a genome-wide basis, in a system that closely models the complex biological and cellular interactions of whole organisms, including the human body. In one aspect, the invention is especially useful in drug development, both because of its ability to validate targets and because of its ability to rapidly identify and to quantify all the expressed genes associated with responses to a potential therapeutic agent.

The invention achieves these goals by exploiting the properties of embryoid bodies. Embryoid bodies represent a complex group of cells differentiating into different tissues. In one embodiment, the cells within an EMBRYOID BODY are substantially synchronized for their differentiation. Accordingly, at known intervals, the majority of the synchronized cells differentiate into the three embryonic germ layers and further differentiate into multiple tissue types, such as cartilage, bone, smooth and striated muscle, and neural tissue, including embryonic ganglia. Thus, the cells within embryoid bodies provide a much closer model to the complexity of whole organisms than do traditional single cell or yeast assays, while still avoiding the cost and difficulties associated with the use of mice and larger mammals. Moreover, the recent availability of human embryoid bodies improves the predictive abilities of the invention by providing an even closer vehicle for modeling toxicity in human organ systems, and in humans.

The embryoid body of the invention comprises a cell population, the majority of which being pluripotent cells capable of developing into different cellular lineages when cultured under appropriate conditions. It is preferred that the embryoid body comprises at least 51% pluripotent cells derived from totipotent ES cells. More preferably, the embryoid body comprises at least 75% pluripotent cells derived from totipotent ES cells. And still more preferably, the embryoid body comprises at least 95% pluripotent cells derived from totipotent ES cells.

In its simplest form, the method of creating a molecular profile according to the present invention involves contacting embryoid bodies with a chemical composition of interest, and then determining the alterations in gene expression, protein expression, or both, in the embryoid body exposed to the chemical composition (the “test embryoid body”) compared to a embryoid body which was not exposed to the agent (a “control embryoid body”).

Furthermore, a library can be generated by compiling molecular profiles for two or more different chemical compositions, such as those having similar toxicities. The molecular profiles of these compositions can be compared with each other, either qualitatively or quantitatively, in order to discern common alterations in their gene or protein expression patterns. For example, while the overall gene or protein expression pattern for each chemical composition may be unique, the changes in expression level of certain specific genes or proteins may be similar among compositions having similar toxicities--some genes/proteins may be similarly up-regulated and therefore expressed in higher amount compared to controls; while other genes/proteins may be similarly down-regulated and therefore expressing in smaller amount compared to controls. These common molecular features of the chemical compositions can then be correlated to their toxicities and serve as surrogate markers for assessing the toxicities of a new or previously untested chemical composition, such as a drug lead in drug screening assays.

Thousands of compounds have undergone preclinical and clinical studies. Preclinical studies include, among other things, toxicity studies in at least two mammalian species, one of which is usually a murine species, typically mice or rats, and clinical trials always include information on any apparent toxicity. A considerable amount of information is available about the toxicity of various of these compounds. Based on the toxicity information available, these compounds can be classified into particular categories of toxicities. For example, a number of chemical compositions are listed in Table 1 according to tissues or organs in which they exet toxicities.

TABLE 1

| | TOXICITIES | | | | | | |
|---|---|---|---|---|---|---|---|
| DRUGS | DEV | LIVER | CV | CNS | BLOOD | INDICATION | TRADE NAMES |
| thalidomide | + | | | | | | |
| methotrexate | + | | | | | antineoplastics | |
| retinoic acid | + | | | | | acne | |
| valproic acid | + | + | | | | seizures | Depakene |
| acetominophen | | + | | | | analgesic | |
| isoniazid | | + | | | | antibiotic | |
| diclofenac (NSAIDS) | | + | | | | anti-inflammatory | Voltarern |
| bromofenac (NSAIDS) | | + | | | | anti-inflammatory | Duract |
| troglitazone | | + | | | | diabetes | Rezulin ™ |
| rosiglitazone | | ntc | | | | diabetes | Avandia ™ |
| trovaflozacin | | + | | | | antibiotic | Trovan ™ |
| ciprofloxacin | | ntc | | | | antibiotic | Cipro ™ |
| erythromycin estolate | | + | | | | antibiotic | |
| pravastatin | | + | | | | lipid lowering | Pravachol ™ |
| atorvastin | | + | | | | lipid lowering | Lipitor ™ |
| clofibrate | | ntc | | | | lipid lowering | Atromid |
| clozapine | | | | | + | antipsychotic | Clozaril |
| chloroamphenicol | | | | | + | antibiotic | Chloromycetin |
| doxorubicin | | | + | | | antineoplastics | |
| daunorubicin | | | + | | | antineoplastics | |
| cyclosophosphamide | | | + | | | antineoplastics | |
| COMPOUNDS | | | | | | | |
| carbon tetrachloride | | + | | | | | |
| cadmium | | + | | | | | |
| phallodidin | | + | | | | | |
| ethanol | | + | | | | | |
| di-methyl formide | | + | | | | | |
| dichlorethylene | | + | | | | | |
| lead | | + | | | | | |
| benzo(a)pyrene | | | + | | | | |
| allylamine | | | + | | | | |
| methylmercury | | | | + | | | |
| trimethyltin | | | | + | | | |
| carbon disulfide | | | | + | | | |
| acrylamide | | | | + | | | |
| hexachloraphene | | | | + | | | |

| | |
|---|---|
| DMSO | not well studied |

“ntc” = non-toxic, limited toxicity, control
“Dev” = developmental
“CV” = cardiovascular
“CNS” central nervous system

In one embodiment of the invention, compositions known for having liver toxicities are used for a systematic analysis of their molecular profiles in EB cells. In another embodiment, compositions causing toxicities to the cardiovascular system are evaluated for their molecular profiles in EB cells. In yet another embodiment of the invention, compositions causing toxicities to the neuronal system are evaluated for their molecular profiles in EB cells. Alternatively, known or potential drugs for treating a disease of choice can be used together in a systematic analysis of their toxicities. In this regard, for example, anti-cancer drugs and drug candidates can be screened for their tissue and organ toxicities.

According to one aspect of the invention, molecular profiles of chemical compositions can be correlated to toxicities these agents demonstrated in non-human animals, in humans, or in both. By then comparing the expression pattern of an embryoid body exposed to a new or previously untested agent to a library of such profiles of expression induced by agents of known toxicity, predictions can be made as to the likely type of toxicity of the new agent. Furthermore, the toxicity of the new agent, if any, can be ranked among the known toxic compositions, providing information for prioritization in drug development.

In addition to its utility in drug development, the invention also has uses in other arenas in which the toxicity of chemical compositions is of concern. Thus, the invention can be utilized to assess the toxicity of agricultural chemicals, such as pesticides and fertilizers. It can further be used with cosmetics. For example, it can be used to screen candidate cosmetics for toxicity prior to moving the compounds into animal studies, thereby potentially reducing the number of animals which need to be subjected to procedures such as the Draize eye irritancy test. Similarly, the methods of the invention can be applied to agents intended for use as “cosmeceuticals,” wherein agents which are primarily cosmetic are also asserted to have some quasi-therapeutic property. Further, the invention can be used to assess the relative toxicity of environmental contaminants, including waste products, petrochemical residues, combustion products, and products of industrial processes. Examples of such contaminants include dioxins, PCBs, and hydrocarbons.

In general, it is preferred that the method used to detect the levels of protein or gene expression provide at least a relative measure of the amount of protein or gene expression. More preferably, the method provides a quantitative measure of protein or gene expression to facilitate the comparison of the protein or gene expression of the embryoid bodies exposed to the test chemical composition to that of embryoid bodies exposed to chemical compositions of known toxicity.

### C. Preparing Embryoid Bodies

In one embodiment, the embryoid bodies used in the present invention can be derived from a population of embryonic stem cells (“ES cells”) under culture conditions allowing differentiation. ES cells are undifferentiated, immature totipotent cells that are capable of giving rise to multiple, specialized cell types and, ultimately, to terminally differentiated cells. ES cells are typically derived from the inner cell mass of early blastocysts, and can be grown indefinitely in culture. See, e.g., Keller et al., WO 96/16162. ES cells are initially totipotent, see, e.g., Hogan, U.S. Pat. No. 5,690,926. Techniques for culturing ES cells are well known in the art. See, e.g., Robertson, E., “Embryo-derived Stem Cell Lines” in Robertson, E. ed., Teratocarcinomas and ES cells: A practical approach, IRL Press (Washington, DC 1987); Hogan, R., et al., eds., Manipulating the Mouse Embryo: A Laboratory Manual, Cold Spring Harbor Laboratory Press, (Cold Spring Harbor, N.Y., 1986).

Methods for preparing mammalian embryoid bodies using ES cells are known in the art. For example, Keller et al., supra, describes preparing EB cell population by culturing ES cells in an embryoid body medium. Typically, ES cells remain at an undifferentiated state in the presence of Leukemia inhibitory factor (“LIF”). LIF is described, for example, in Gearing, U.S. Pat. No. 5,187,077. In vitro propagation of ES cells using LIF is taught in Williams, U.S. Pat. No. 5,166,065.

To commence differentiation, ES cells are removed from the LIF-containing embryonic stem cell medium and re-cultured in medium which does not contain LIF. See, Keller, et al., supra, at 13. Generally, the cells are cultured in plasticware which has not been treated to promote adherence (such as bacterial-grade plasticware, Teflon™ coated plasticware, or other materials known to decrease adherence). The cells then tend to bunch up, and the interaction of the ES cells as a mass acts to induce the formation of embryoid bodies, which commence differentiating into the three germ layers and further into cells of particular tissue types, such as muscle cells, epithelial cells, neuronal cells, and hematopoietic cells. Snodgrass, et al., “Embryonic Stem Cells: Research and Clinical Potentials” in Smith and Sacher, eds. Peripheral Blood Stem Cells American Association of Blood Banks, Bethesda Md. (1993).

Thomson, WO 96/22362, describes a primate ES cell population that remains undifferentiated state indefinitely in the presence of fibroblast feeder cells. Feeder cells are cells which have been irradiated to remove their ability to divide, but which provide a substrate and various factors supporting the culturing of ES cells. See, e.g., Robertson, supra, and Hogan, et al., supra. Primary mouse embryo fibroblast cells are preferred, although mouse 3T3 or STO cells can be used. E.g., Hogan, et al., supra; Tadaro and Green (1963) J. Cell Biol. 17:299; Ware and Axelrad (1972) Virology 50:339. Upon removal from the feeder cells, the primate ES cells will differentiate into various cell types and, when grown at high densities, form embryoid bodies. See, Thomson, supra; Thomson et al. (1996) Biol. Reprod. 57:254-259; and Thomson and Marshall (1998) Curr Top Dev Biol. 38:133-165. Formation of embryoid bodies from ES cells of numerous other mammals, such as pigs, have also been reported. See, Shim, et al. (1997) Biol. Reprod. 57:1089-95.

Embryoid bodies obtained according to the present invention can be identified visually by their morphology, as known in the art and described in Keller et al, supra. Under defined culturing conditions, an embryoid body has a general morphology of tightly packed cells or cell aggregate or cell mass, in which individual cells are not easily detectable. The number of cells in an embryoid body, which can be estimated by the size of the cell mass and the approximate size of individual cells, can range from about 5 to about 2,000, although preferably from about 10 to about 100. An even more preferred number of cells in an embryoid body is about 20.

Alternatively, the embryoid bodies obtained according to the present invention can be identified by the detection of specific markers such as antibodies specific to a population of embryoid body cells at defined stage. For example, Keller et al, supra, describes that a Day-4 EB cell population expresses substantially low amounts of Sca-1, C-kit receptor and Class I H-2b and essentially no Thy 1, VLA-4, CD44 and CD45. Thus, the cells in a Day-4 EB have substantially the same staining pattern when such cells are stained with antibodies to these surface antigens.

If necessary, embryoid bodies obtained and cultured according to the present invention may be isolated from the culture based on their physical or chemical properties (such as size, mass, density, specific antigen or gene expression), using methods known in the art (such as flow cytometry, cell sorting, filtration or centrifugation).

In a widely noted recent development, two groups have reported the development of ES cells from human blastocysts. See, Thomson et al. (1998) Science 282:1145-1147 and Shamblott, et al. (1998) Proc Natl Acad Sci (USA) 95:13726-13731.

In Thomson et al.'s work, human embryos produced by in vitro fertilization for clinical purposes were donated by individuals after informed consent and institutional review board approval. The embryos were cultured to the blastocyst stage, inner masses isolated, and ES cell lines obtained by essentially the same means previously described (and referenced above) for nonhuman primate ES cells. Id. The cells were capable of differentiating into derivatives of all three embryonic germ layers., Id. As with other primate ES cells, LIF was not sufficient to keep the human ES cells from differentiating in the absence of fibroblast feeder cells, but differentiated even in the presence of fibroblast feeder cells when grown to confluence and allowed to pile up in the culture dish. Id.

In Shamblott et al.'s work, gonadal ridges and mesenteries containing primordial germ cells (“PGCs”), taken from human embryos obtained from terminated pregnancies 5-9 weeks postfertilization, were cultured on mouse STO fibroblast feeder layers in the presence of human recombinant LIF, human recombinant basic fibroblast growth factor, and forskolin. Over a period of 7-21 days, the PGCs gave rise to colonies of stem cells which developed into embryoid bodies. The embryoid bodies were shown to contain a wide variety of differentiated cell types, including derivatives of all three embryonic germ layers. It is expected that human embryoid bodies such as those created by Thomson et al. and Shamblott et al. can be used in the methods of the invention.

ES cells can also be formed from enucleated cells into which the nucleus of a desired human or mammalian cell has been inserted. See, e.g., Robl, et al., International Publication Number WO 98/07841.

The embryoid bodies used to test the chemical composition can be of any vertebrate species. The choice of the particular species from which the embryoid body is derived will typically reflect a balance of several factors. First, depending on the purpose of the study, one or more species may be of particular interest. For example, human embryoid bodies will be of particular interest for use with compositions being tested as potential human therapeutics, while equine, feline, bovine, porcine, caprine, canine, or sheep embryoid bodies may be of more interest for a potential veterinary therapeutic.

Second, even with respect to testing of human therapeutics, cost and handling considerations may dictate that some or all testing be performed with non-human, and even non-primate embryoid bodies. Obtaining human ES cells, for example, currently requires not only informed consent and institutional review board review, but also very labor intensive tending. See, Marshall, Science 282:1014-1015 (Nov. 6, 1998). Obtaining primate embryoid bodies, while obviously not entailing the same legal requirements, requires first obtaining the primates, and entails significant and costly animal husbandry obligations. Accordingly, for much testing, it may be desirable to use embryoid bodies from mice, rats, guinea pigs, rabbits, and other readily available, and less expensive, laboratory animals.

Third, it will often be of value to select a species as to which considerable information is available on the toxicity of chemical compositions, so that observed changes in gene and protein expression can be correlated to various types of toxicity. For this reason, mice and rats are preferred embodiments. Most pre-clinical testing is performed on at least one murine species, and there therefore exists a large body of information on the toxicity of various compounds on various tissues of mice and on rats. Using embryoid bodies derived from mice or rats permits the correlation of the alterations in gene or protein expression in the embryoid bodies with the toxicities exhibited by these agents in those species. Embryoid bodies of other species commonly used in preclinical testing, such as guinea pigs, rabbits, pigs, and dogs, are also preferred for the same reason. Typically, embryoid bodies of these species will be used for “first pass” screening, or where detailed information on toxicity in humans is not needed, or where a result in a murine or other one of these laboratory species has been correlated to a known toxicity or other effect in humans.

Fourth, although primates are not as widely used in preclinical testing and are often more expensive to purchase and to maintain than other laboratory animals, their biochemistry and developmental biology is considerably closer to that of humans than those of the more common laboratory animals. Embryoid bodies derived from primates is therefore preferred for toxicity testing where the study is sufficiently important to justify the additional cost and handling considerations. Most preferred are human embryoid bodies, since conclusions about the toxicity of agents in these embryoid bodies can be considered the most directly relevant to the effect of a chemical composition on humans. It is anticipated that studies in primate or human embryoid bodies will be performed to confirm results of toxicity studies in embryoid bodies of other species. It is anticipated that human embryoid bodies will be used where toxicity in humans is of sufficient interest to warrant undertaking the cost and legal hurdles, and will become more preferred over time as the legal barriers to the use of human ES cells become less onerous.

Fifth, with respect to human therapeutics, regulatory agencies generally require animal data before human trials can begin; it will generally be desirable to use embryoid bodies of species which will be used in the preclinical animal studies. The results of toxicity testing in the embryoid bodies can then guide the researcher on the degree and type of toxicity to anticipate during the animal trials. Certain animal species are known in the art to be better models of human toxicity of different types than are others, and species also differ in their ability to metabolize drugs. See, e.g., Williams, Environ Health Perspect. 22:133-138 (1978); Duncan, Adv Sci 23:537-541 (1967). Thus, the particular species preferred for use in a particular preclinical toxicity study may vary according to the intended use of the drug candidate. For example, a species which provide a suitable model for a drug intended to affect the reproductive system may not be as suitable a model for a drug intended to affect the nervous system. Criteria for selecting appropriate species for preclinical testing are well known in the art.

While ES cells from different species can be used in the methods of the invention, in general, mammalian cells are preferred. In the discussions below, it is assumed that in any given comparison of control and test embryoid bodies, the embryoid bodies used as controls and those used to test the effects of the chemical compositions are derived from ES cells of the same species.

D. Contacting Embryoid Bodies with Chemical Compositions

1. General

Once an embryoid body culture has been initiated, it can be contacted with a chemical composition. Conveniently, the chemical composition is in an aqueous solution and is introduced to the culture medium. The introduction can be by any convenient means, but will usually be by means of a pipette, a micropipettor, or a syringe. In some applications, such as high throughput screening, the chemical compositions will be introduced by automated means, such as automated pipetting systems, which may be on robotic arms. Chemical compositions can also be introduced into the medium as in powder or solid forms, with or without pharmaceutical excipients, binders, and other materials commonly used in pharmaceutical compositions, or with other carriers which might be employed in the intended use. For example, chemical compositions intended for use as agricultural chemicals or as petrochemical agents can be introduced into the medium by themselves to test the toxicity of those chemicals or agents, or introduced in combination with other materials with which they might be used or which might be found in the environment, to determine if the combination of the chemicals or agents has a synergistic effect. Typically, the cultures will be shaken at least briefly after introduction of a chemical composition to ensure the composition is dispersed throughout the medium.

2. Timing of Contacting

The time as which a chemical composition is added to the culture is within the discretion of the practitioner and will vary with the particular study objective. Conveniently, the chemical composition will be added as soon as the embryoid body develops from the stem cells, permitting the determination of the alteration in protein or gene expression on the development of all the tissues of the embryoid body. It may be of interest, however, to focus the study on the effect of the composition on a particular tissue type. As previously noted, individual tissues, such as muscle, nervous, and hepatic tissue, are known to develop at specific times after the embryoid body has formed. Addition of the chemical composition can therefore be staged to occur at the time the tissue of interest commences developing, or at a chosen time after commencement of that development, in order to observe the effect on altering gene or protein expression in the tissue of interest.

3. Dosing of the Chemical Composition

Different amounts of a chemical composition will be used to contact an embryoid body depending on the amount of information known about the cytotoxicity of that composition, the purposes of the study, the time available, and the resources of the practitioner. A chemical composition can be administered at just one concentration, particularly where other studies or past work or field experience with the compound have indicated that a particular concentration is the one which is most commonly found in the body. More commonly, the chemical composition will be added in different concentrations to cultures of embryoid bodies run in parallel, so that the effects of the concentration differences on gene or protein expression and, hence, the differences in toxicity of the composition at different concentrations, can be assessed. Typically, for example, the chemical composition will be added at a normal or medium concentration, and bracketed by twofold or fivefold increases and decreases in concentration, depending on the degree of precision desired.

Where the composition is one of unknown cytotoxicity, a preliminary study is conveniently first performed to determine the concentration ranges at which the composition will be tested. A variety of procedures for determining concentration dosages are known in the art. One common procedure, for example, is to determine the dosage at which the agent is directly cytotoxic. The practitioner then reduces the dose by one half and performs a dosing study, typically by administering the agent of interest at fivefold or twofold dilutions of concentration to parallel cultures of cells of the type of interest. For environmental contaminants, the composition will usually also be tested at the concentration at which it is found in the environment. For agricultural chemicals, such as pesticides which leave residues on foodstuffs, the agent will usually be tested at the concentration at which the residue is found, although it will likely be tested at other concentrations as well.

E. Detecting Alterations in Levels of Gene or Protein Expression

1. Detecting Protein Expression Alterations

Protein expression can be detected by a number of methods known in the art. For example, the proteins in a sample can be separated by sodium dodecyl sulphate-polyacrylamide gel electrophoresis (“SDS-PAGE”) and visualized with a stain such as Coomassie blue or a silver stain. Radioactive labels can be detected by placing a sheet of X-ray film over the gel. Proteins can also be separated on the basis of their isoelectric point via isoelectric focusing, and visualized by staining. Further, SDS-PAGE can be performed in combination with isoelectric focusing (usually performed in perpendicular directions) to provide two-dimensional separation of the proteins in a sample. Proteins can further be separated by such techniques as high pressure liquid chromatography, FPLC, thin layer chromatography, affinity chromatography, gel-filtration chromatography, ion exchange chromatography, surface enhanced laser desorption/ionization (“SELDI”), matrix-assisted laser desorption/ionization (“MALDI”), and, if the sedimentation rates are sufficiently different, density gradient centrifugation. Detecting alterations in levels of protein expression using these techniques can be accomplished, for example, by running in parallel samples from embryoid bodies contacted with a chemical composition whose effect is of interest (“test samples”) and samples from embryoid bodies cultured under identical conditions except for the presence of the chemical composition of interest (“control samples”), and noting any differences in the proteins detected and the amount of the proteins detected.

Immunodetection provides a group of useful techniques for detecting alterations in protein expression. In these techniques, antibodies are typically raised against the protein by injecting the protein into mice or rabbits following standard protocols, such as those taught in Harlow and Lane, Antibodies, A Laboratory Manual(Cold Spring Harbor Laboratory, Cold Spring Harbor, N.Y., 1988). The antibodies so raised can then be used to detect the presence of and quantitate the protein in a variety of immunological assays known in the art, such as ELISAs, fluorescent immunoassays, Western and dot blots, immunoprecipitations, and focal immunoassays. Alterations in protein expression can be determined by running parallel tests on test and control samples and noting any differences in results between the samples. Results of ELISAs, for example, can be directly related to the amount of protein present.

Tagging provides another way to detect and determine changes in protein expression. For example, the gene encoding the protein can be engineered to produce a hybrid protein containing a detectable tag, so that the protein can be specifically detected by detection of the tag. Systems are available which permit the direct imaging and quantitation of radioactive labels in, for example, gels on which the proteins have been separated. Differences in expression can be determined by observing differences in the amount of the tag present in test and control samples.

Proteins can also be analyzed by standard protein chemistry techniques. For example, proteins can be analyzed by performing proteolytic digests with trypsin, Staphylococcus B protease, chymotrypsin, or other proteolytic enzymes. Differences in expression can be determined by comparing relative amounts of the digested products.

One particularly preferred method for determining differences in protein expression is mass spectroscopy, or “MS,” which provides the broadest profile of the broadest number of proteins for the least effort. Moreover, MS permits not only accurate detection of proteins present in a sample, but also quantitation. The procedure can be used either by itself, or in combination with one or more of the preceding methods based on selective physical properties to partition the proteins present in a sample. Partitioning reduces the number of proteins of different physical properties in the sample and results in a better MS analysis by permitting a comparison of proteins of similar size, electrostatic charge, affinity for metal ions, or the like. Thus, for example, the proteins in a sample can be subjected to SDS-PAGE and isoelectric focusing, and a resulting spot of interest on the gel can then be subjected to MS. In Example 1, below, initial partitioning was performed using a sizing column and a second partitioning was performed using SELDI. It should be noted that, in the protocol followed in Example 1, the proteins with molecular weights smaller than 30 kD were analyzed. Alternatively, of course, the higher weight proteins could be analyzed in the methods of the invention, and the proteins do not need to be fractionated if the practitioner is prepared to analyze all the proteins in a sample or, for example, if a preliminary analysis shows that the total number of different proteins in a sample is small enough to be analyzed without partitioning.

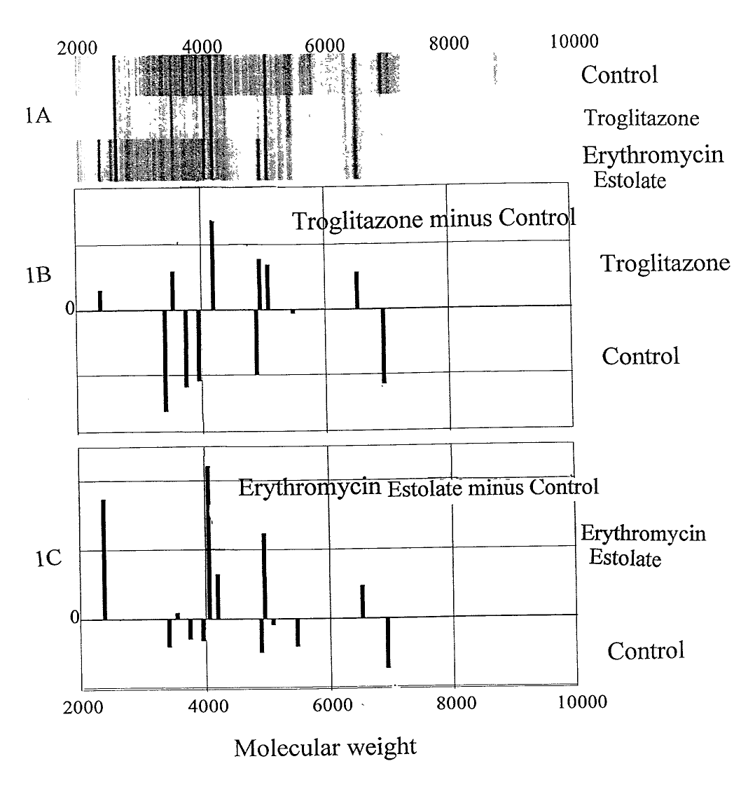

Computers attached to the mass spectrometer can also be used to analyze the samples to facilitate determination of whether a change in protein expression may be indicative of a particular toxicity. For example, the readout from the MS can be used in a “subtractive calculation” in which the protein expression in control embryoid bodies is quantitated and then subtracted from the quantitated protein expression of embryoid bodies contacted with a chemical composition, with only the proteins expressed in greater or lesser quantities than those expressed by the control embryoid bodies being shown. This method immediately focuses attention on differences in protein expression between a control and a test population. Examples of such comparisons are shown in FIGS. 1B and 1C and discussed in detail in Example 1, below.

2. Detecting Gene Expression Alterations

A number of methods are known in the art for detecting and comparing levels of gene expression.

One standard method for such comparisons is the Northern blot. In this technique, RNA is extracted from the sample and loaded onto any of a variety of gels suitable for RNA analysis, which are then run to separate the RNA by size, according to standard methods (see, e.g., Sambrook, J., et al., Molecular Cloning, A Laboratory Manual. Cold Spring Harbor Laboratory Press, Cold Spring Harbor, N.Y. (2nd ed. 1989)). The gels are then blotted (as described in Sambrook, supra), and hybridized to probes for RNAs of interest. The probes can be radioactive or non-radioactive, depending on the practitioner's preference for detection systems. For example, hybridization with the probe can be observed and analyzed by chemiluminescent detection of the bound probes using the “Genius System,” (Boehringer Mannheim Corporation, Indianapolis, Ind.), following the manufacturer's directions. Equal loading of the RNA in the lanes can be judged, for example, by ethidium bromide staining of the ribosomal RNA bands. Alternatively, the probes can be radiolabeled and detected autoradiographically using photographic film.

The RNA can also be amplified by any of a variety of methods and then detected. For example, Marshall, U.S. Pat. No. 5,686,272, discloses the amplification of RNA sequences using ligase chain reaction, or “LCR.” LCR has been extensively described by Landegren et al., Science, 241:1077-1080 (1988); Wu et al., Genomics, 4:560-569 (1989); Barany, in PCR Methods and Applications, 1:5-16 (1991); and Barany, Proc. Natl. Acad. Sci. USA, 88:189-193 (1991). Or, the RNA can be reverse transcribed into DNA and then amplified by LCR, polymerase chain reaction (“PCR”), or other methods. An exemplar protocol for conducting reverse transcription of RNA is taught in U.S. Pat. No. 5,705,365. Selection of appropriate primers and PCR protocols are taught, for example, in Innis, M., et al., eds., PCR Protocols 1990 (Academic Press, San Diego CA) (hereafter “Innis et al.”). Differential expression of messenger RNA can also be compared by reverse transcribing mRNA into cDNA, which is then cleaved by restriction enzymes and electrophoretically separated to permit comparison of the cDNA fragments, as taught in Belyavsky, U.S. Pat. No. 5,814,445.

Typically, primers are labeled at the 5′ terminus with biotin or with any of a number of fluorescent dyes. Probes are usually labeled with an enzyme, such as horseradish peroxidase (HRP) and alkaline phosphatase, see, Levenson and Chang, Nonisotopically Labeled Probes and Primers in Innis, et al., supra, but can also be labeled with, for example, biotin-psoralen. Detailed example protocols for labeling primers and for synthesizing enzyme-labeled probes are taught by Levenson and Chang, supra. Or, the probes can also be labeled with radioactive isotopes. An exemplar protocol for synthesizing radioactively labeled DNA and RNA probes is set forth in Sambrook et al., supra. Usually, ³²P is used for labeling DNA and RNA probes. A number of methods for detection of PCR products are known. See, e.g., Innis, supra, which sets forth a detailed protocol for detecting PCR products using non-isotopically labeled probes. Generally, there is a step permitting hybridization of the probe and the PCR product, following which there are one or more development steps to permit detection.

For example, if a biotinylated psoralen probe is used, the hybridized probe is incubated with streptavidin HRP conjugate and then incubated then incubated with a chromogen, such as tetramethylbenzidine (TMB). Alternatively, if the practitioner has chosen to employ a radioactively labeled probe, PCR products to which the probe has hybridized can be detected by autoradiography. As another example, biotinylated dUTP (Bethesda Research Laboratories, MD) can be used during amplification. The labeled PCR products can then be run on an agarose gel, Southern transferred to a nylon filter, and detected by, for example, a streptavidin/alkaline phosphatase detection system. A protocol for detecting incorporated biotinylated dUTP is set forth, e.g., in Lo et al., Incorporation of Biotinylated dUTP, in Innis et al., supra. Finally, the PCR products can be run on agarose gels and nucleic acids detected by a dye, such as ethidium bromide, which specifically recognizes nucleic acids.

Sutcliffe, U.S. Pat. No. 5,807,680, teaches a method for the simultaneous identification of differentially expressed mRNAs and measurement of relative concentrations. The technique, which comprises the formation of cDNA using anchor primers followed by PCR, allows the visualization of nearly every mRNA expressed by a tissue as a distinct band on a gel whose intensity corresponds roughly to the concentration of the MRNA.

Another group of techniques employs analysis of relative transcript expression levels. Four such approaches have recently been developed to permit comprehensive, high throughput analysis. First, CDNA can be reverse transcribed from the RNAs in the samples (as described in the references above), and subjected to single pass sequencing of the 5′ and 3′ ends to define expressed sequence tags for the genes expressed in the test and control samples. Enumerating the relative representation of the tags from the different samples provides an approximation of the relative representation of the gene transcript within the samples.

Second, a variation on ESTs has been developed, known as serial analysis of gene expression, or “SAGE,” which allows the quantitative and simultaneous analysis of a large number of transcripts. The technique employs the isolation of short diagnostic sequence tags and sequencing to reveal patterns of gene expression characteristic of a target function, and has been used to compare expression levels, for example, of thousands of genes in normal and in tumor cells. See, e.g., Velculescu, et al., Science 270:368-369 (1995); Zhang, et al., Science 276:1268-1272 (1997).

Third, approaches have been developed based on differential display. In these approaches, fragments defined by specific sequence delimiters can be used as unique identifiers of genes, when coupled with information about fragment length within the expressed gene. The relative representation of an expressed gene within a cell can then be estimated by the relative representation of the fragment associated with that gene. Examples of some of the several approaches developed to exploit this idea are the restriction enzyme analysis of differentially-expressed sequences (“READS”) employed by Gene Logic, Inc., and total gene expression analysis (“TOGA”) used by Digital Gene Technologies, Inc. CLONTECH, Inc. (Palo Alto, Calif.), for example, sells the Delta™ Differential Display Kit for identification of differentially expressed genes by PCR.

Fourth, in preferred embodiments, the detection is performed by one of a number of techniques for hybridization analysis. In these approaches, RNA from the sample of interest is usually subjected to reverse transcription to obtain labeled cDNA. The cDNA is then hybridized, typically to oligonucleotides or cDNAs of known sequence arrayed on a chip or other surface in a known order. The location of the oligonucleotide to which the labeled cDNA hybridizes provides sequence information on the cDNA, while the amount of labeled hybridized RNA or cDNA provides an estimate of the relative representation of the RNA or cDNA of interest. Further, the technique permits simultaneous hybridization with two or more different detectable labels. The hybridization results then provide a direct comparison of the relative expression of the samples.

A number of kits are commercially available for hybridization analysis. These kits allow identification of specific RNA or cDNAs on high density formats, including filters, microscope slides, microchips, and technologies relying on mass spectrometry. For example, Affymetrix, Inc. (Santa Clara, Calif.), markets GeneChip™ Probe arrays containing thousands of different oligonucleotide probes with known sequences, lengths, and locations within the array for high accuracy sequencing of genes of interest. CLONTECH, Inc.'s (Palo Alto, Calif.) Atlas™ cDNA Expression Array permits monitoring of the expression patterns of 588 selected genes. Hyseq, Inc.'s (Sunnyvale, Calif.) Gene Discovery Module permits high throughput screening of RNA without previous sequence information at a resolution of 1 mRNA copy per cell. Incyte Pharmaceuticals, Inc. (Palo Alto, Calif.) offers microarrays containing, for example, ordered oligonucleotides of human cancer and signal transduction genes. Techniques used by other companies in the field are discussed in, e.g., Service. R., Science 282:396-399 (1998)

3. Labels

Both proteins and genes can be labeled to detect the alteration in levels of expression in the methods of the invention. The term “label” refers to a composition detectable by spectroscopic, photochemical, biochemical, immunochemical, or chemical means. For example, useful nucleic acid and protein labels include 32P, ³⁵S, fluorescent dyes, electron-dense reagents, enzymes (e.g., as commonly used in an ELISA), biotin, dioxigenin, or haptens and proteins for which antisera or monoclonal antibodies are available.

A wide variety of labels and conjugation techniques are known and are reported extensively in both the scientific and patent literature, and are generally applicable to the present invention for the labeling of nucleic acids, amplified nucleic acids, and proteins. Suitable labels include radionucleotides, enzymes, substrates, cofactors, inhibitors, fluorescent moieties, chemiluminescent moieties, magnetic particles, and the like. Labeling agents optionally include e.g., monoclonal antibodies, polyclonal antibodies, proteins, or other polymers such as affinity matrices, carbohydrates or lipids. Detection of labeled nucleic acids or proteins may proceed by any of a number of methods, including immunoblotting, tracking of radioactive or bioluminescent markers, Southern blotting, Northern blotting, or other methods which track a molecule based upon size, charge or affinity. The particular label or detectable moiety used and the particular assay are not critical aspects of the invention.

The detectable moiety can be any material having a detectable physical or chemical property. Such detectable labels have been well developed in the field of gels, columns, and solid substrates, and in general, labels useful in such methods can be applied to the present invention. Thus, a label is any composition detectable by spectroscopic, photochemical, biochemical, immunochemical, electrical, optical or chemical means. Useful labels in the present invention include fluorescent dyes (e.g., fluorescein isothiocyanate, Texas red, rhodamine, and the like), radiolabels (e.g., ³H, ¹²⁵I, ³⁵S, ¹⁴C, or ³²p), enzymes (e.g., LacZ, CAT, horse radish peroxidase, alkaline phosphatase and others, commonly used as detectable enzymes, either as marker gene products or in an ELISA), nucleic acid intercalators (e.g., ethidium bromide) and calorimetric labels such as colloidal gold or colored glass or plastic (e.g. polystyrene, polypropylene, latex, etc.) beads, as well as electronic transponders (e.g., U.S. Pat. No. 5,736,332).

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule. Bruchez et al. (1998) Science 281: 2013-2016. Similarly, highly fluorescent quantum dots (zinc sulfide-capped cadmium selenide) have been covalently coupled to biomolecules for use in ultrasensitive biological detection. Warren and Nie (1998) Science 281: 2016-2018.

The label is coupled directly or indirectly to the desired nucleic acid or protein according to methods well known in the art. As indicated above, a wide variety of labels may be used, with the choice of label depending on the sensitivity required, ease of conjugation of the compound, stability requirements, available instrumentation, and disposal provisions. Non-radioactive labels are often attached by indirect means. Generally a ligand molecule (e.g., biotin) is covalently bound to a polymer. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with labeled anti-ligands. Alternatively, any haptenic or antigenic compound can be used in combination with an antibody.

Labels can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, fluorescent green protein, and the like. Chemiluminescent compounds include luciferin, and 2,3-dihydrophthalazinediones, e.g., luminol.

Means of detecting labels are well known to those of skill in the art. Thus, for example, where the label is a radioactive label, means for detection include a scintillation counter, proximity counter (microtiter plates with scintillation fluid built in), or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDS) or photomultipliers and the like. Similarly, enzymatic labels may be detected by providing appropriate substrates for the enzyme and detecting the resulting reaction product. Finally simple calorimetric labels are often detected simply by observing the color associated with the label. Thus, in various dipstick assays, conjugated gold often appears pink, while various conjugated beads appear the color of the bead.

F. Correlating Molecular Profiles With Toxicities

The invention contemplates multiple iterations of compiling a library of molecular profiles by contacting test embryoid bodies with an ever-widening group of chemical compositions having predetermined toxicities. The toxicities and biological effects of many chemical compositions are already known through previous animal or clinical testing. Any such information is carefully noted along with the alterations of gene or protein expression in embryoid bodies. As the data from tests on a number of chemical compositions, or agents, is gathered, it is assembled to form a library. Separate libraries can be maintained for each type of toxicity; preferably, a single database can be maintained recording the results of all the tests conducted and any available toxicity information on the agents to which the embryoid bodies were exposed. Preferably, biological effects are also noted. Past experience has indicated that biological effects often become associated with, or markers for, particular toxicities as the biology of the toxicity becomes better understood.

The invention contemplates that each iteration of contacting test embryoid bodies with a chemical composition will generate a pattern of gene or protein expression, or both, characteristic for that chemical composition. The determination of the alteration in gene or protein expression of a reasonably large number of chemical compounds of similar toxicity is desirable so that patterns of gene or protein expression, or both, associated with that toxicity can be determined. Changes in gene or protein expression patterns in EB cells that are common to classes of drugs that have similar toxicities will serve as surrogate molecular profiles useful for recognizing compounds that are likely to have related biology and toxicities. It is the correlation of these alterations in gene or protein expression and toxicities that gives the invention its predictive power with respect to previously untested compounds.

The correlation of patterns of gene or protein expression with toxicities can be performed by any convenient means. For example, visual comparisons of patterns can be performed to determine patterns associated with different types of toxicities. More conveniently, the correlation can be done by computer, using one of the database programs discussed in the previous section. Preferably, the correlation is performed by a computer using a neural network program, since neural network programs are specifically designed for pattern recognition. Once a correlation of expression markers which are biomarkers for a particular toxicity has been made, a comparison can be made, again conveniently by computer, of known patterns to the pattern of gene or protein expression induced by a new or unknown chemical composition to provide the closest matches of expression. The patterns can then be reviewed to predict the likely toxicity of the new or unknown chemical.

G. Typing and Ranking Yoxicities of Test Chemical Compositions

A molecular profile of a test chemical composition can be established by detecting the alterations in gene or protein expression in embryoid bodies contacted by the test chemical composition as described in previous sections. Once the molecular profile of the test composition is determined, it can be compared to that of a chemical composition with predetermined toxicities or, preferably, to a library of molecular profiles of chemical compositions with predetermined toxicities. The outcome of such comparison provide information for one to predict the likelihood of whether the test composition is toxic, what type of toxicities, and how toxic it would be as compared to the other known toxic compositions.

For the purpose of practicing the invention, the predictions of toxicity of the test composition based on its molecular profiles in EB cells does not have to be 100% accurate.

To have a major positive impact on the efficiency and costs of drug development, one only has to modestly increase the probability that the less toxic and thus more successful drug candidates are, for example, on the top half of a prioritized list of new drug leads.

As noted in previous sections, alterations in gene or protein expression in embryoid bodies exposed to a chemical composition can be detected by any of a number of means known in the art. Protein expression determined by MS is particularly convenient for such comparisons since the output data is typically fed directly into a computer connected to the mass spectrometer and is immediately available for a variety of calculations. If the alterations are susceptible to graphical representation, as when MS is used as the means of detection, a direct comparison can be made of the effect of the chemical composition on the expression of proteins compared to the control embryoid bodies. If the alterations are detected by, for example, an ELISA, which produces a numerical readout, then the numerical readouts can be used to quantitate the expression of the protein. For gene expression, Northern blots can be correlated to the amount of RNA present for each RNA probed. Where gene expression is detected by hybridization arrays, the pattern of hybridization for nucleic acids from the test and control embryonic bodies provides a basis for comparison.

The comparison of molecular profiles can be done by a number of means known in the art. Usually, the graphs resulting from the calculations can be stored, for example, in file folders or the like, and examined visually to discern common patterns of expression compared to the control, as well as differences. Conveniently, however, the data can be stored on and compared by a computer. Programs are available, for example, to compare mass spectrometry data. FIGS. 1B and 1C, for example, demonstrate the use of “subtractive calculation” and graphical representation to compare protein expression in the control embryoid bodies (“control samples”) against that of the embryoid bodies contacted with either of two chemical compositions (“test samples”). In this comparison, the amount of each protein expressed by the control samples is subtracted from the amount expressed by the test samples. The control sample value is represented by a horizontal line, and any protein expressed in a different amount is represented as a line above or below the line (representing positive and negative amounts compared to the control, respectively), with the height of the line designating the amount by which the expression of the test sample is different from that of the control. This method focuses attention on the differences in protein expression. In a like manner, the program can also be used to compare the expression of two or more test samples so that any differences in expression patterns can be readily discerned. It is expected that the more similar the pattern of expression, the more similar will be the effect, and the type of toxicity, of the two agents.

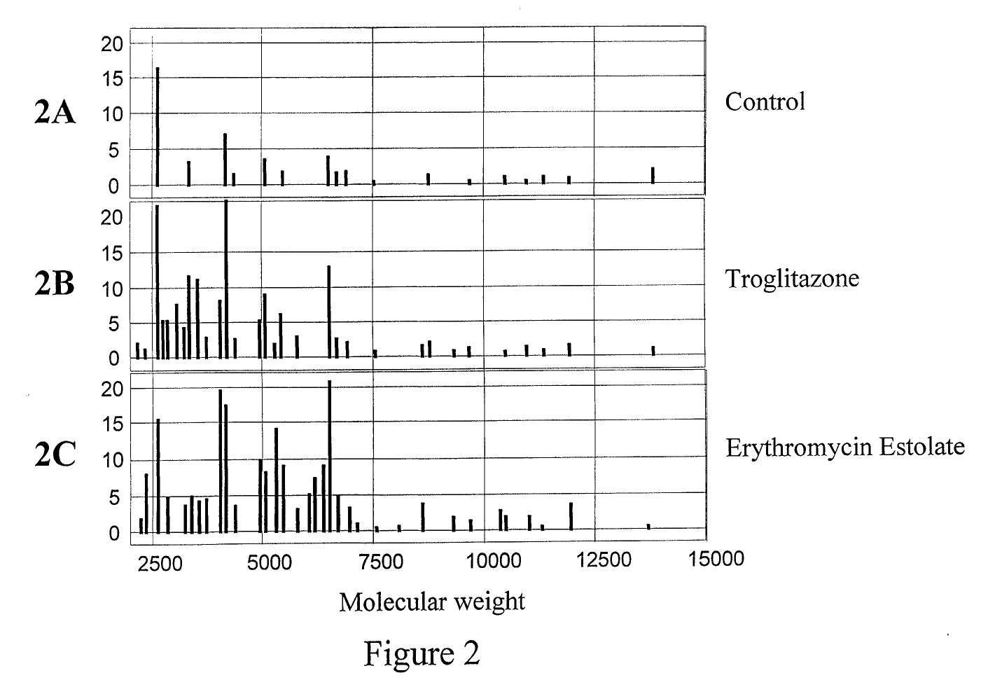

Another form of comparison is shown in FIGS. 2, 3, and 4. These figures graphically depict the small nuclear, small cytoplasmic, and large cytoplasmic proteins expressed by control samples and by test samples exposed to one of two chemical compositions, as well the amount of the protein expressed by the samples. These graphs can be compared visually, and the proteins and the amounts expressed recorded manually. Preferably, the results are placed into a computer database, with information about the known toxicities of the chemical compositions recorded in searchable data fields. Entries of data from other forms of detecting alterations in protein or gene expression can also be reviewed and recorded manually or in a computer database. For example, the values from an ELISA, or the proteins identified on a Western blot can be recorded to identify the types and amounts of proteins expressed in control and test samples. Similarly, the patterns on a Northern blot, or the hybridization pattern on an oligonucleotide array, can be recorded to identify the gene expression of control and test samples. The information can be kept manually, but preferably is maintained in a computer searchable form.

Standard database programs, such as Enterprise Data Management (Sybase, Inc., Emeryville, Calif.) or Oracle8 (Oracle Corp., Redwood Shores, Calif.) can be used to store and compare information. Alternatively, the data can be recorded, or analyzed, or both, in specifically designed programs available, for example, from Partek Inc. (St. Charles, Mo.).

Additionally, companies selling integrated analytical systems, such as mass spectrometers, provide with the machines integrated software for recording results. Such companies include Finnigan Corp. (San Jose, Calif.), Perkin-Elmer Corp. (Norwalk Conn.), Ciphergen Biosystems, Inc. (Palo Alto Calif.), and Hewlett Packard Corp. (Palo Alto, Calif.). Similarly, companies such as Incyte Pharmaceuticals, Inc. (Palo Alto Calif.) providing oligonucleotide hybridization services maintain proprietary image recognition algorithms to record and analyze the scanned images of hybridization arrays.

In a preferred embodiment, the data can be recorded and analyzed by neural network technology. Neural networks are complex non-linear modeling equations which are specifically designed for pattern recognition in data sets. One such program is the NeuroShell Classifier™ classification algorithm from Ward Systems Group, Inc. (Frederick, Md.). Other neural network programs are available from, e.g., Partek, Inc., BioComp Systems, Inc. (Redmond Wash.) and Z Solutions, LLC (Atlanta, Ga.).

H. Adapting Array Readers

In one embodiment, the invention relates to the formation of arrays of hybridized oligonucleotides or of bound proteins to detect changes in gene or protein expression, respectively. Such arrays can be scanned or read by array readers.

Typically, the array reader will have an optical scanner adapted to read the pattern of labels on an array, such as of bound proteins or hybridized oligonucleotides, operably linked to a computer which has stored on it, or accessible to it (for example, on an external drive or through the internet) one or more data files having a plurality of gene expression or protein expression profiles of mammalian embryoid bodies contacted with known or unknown toxic chemical compositions. The array reader can, however, be adapted with a detection device suitable to “read” labels that can not be read optically, such as electronic transponders.

I. Use in High Throughput Screening

The methods of the invention can be readily adapted to high throughput screening. High throughput (“HTP”) screening is highly desirable because of the large number of uncharacterized compounds already developed in the larger pharmaceutical companies, as well as the flood of new compounds now being synthesized by combinatorial chemistry. Using the invention, hundreds of chemical compositions can be tested on embryoid bodies and the resulting alterations in gene or protein expression, or both, compared to toxicities of known chemical compositions to predict the type and possibly the degree of toxicity the new compounds possess. Those compositions with acceptable toxicity profiles can then be considered for further levels of testing.

HTP screening can be facilitated by using automated and integrated culture systems, sample preparation (protein or RNA/cDNA), and analysis. These steps can be performed in regular labware using standard robotic arms, or in more recently developed microchip and microfluidic devices, such as those developed by Caliper Technologies Corp. (Palo Alto, Calif.), described in U.S. Pat. No. 5,800,690, by Orchid Biocomputer, Inc. (Princeton, N.J.), described in the Oct. 25, 1997 New Scientist, and by other companies, which provide methods of automated analysis using very low volumes of reagents. See, e.g., McCormick, R., et al., Anal. Chem. 69:2626-2630 (1997); Turgeon, M., Med Lab. Management Rept, Dec. 1997, page 1.

### EXAMPLES

### Example 1

### Selecting Chemical Compounds for Toxicity Screening

Compositions that fall into particular categories of toxicity are used to establish molecular profiles and compile libraries for particular toxicities. Table 1 lists a number of compositions that are known to be toxic to certain tissues or organs or during developmental stages. In particular, those compositions causing liver toxicities are assessed for their molecular profiles by determining alterations of gene or protein expression patterns in embryoid bodies contacted by each composition. A library comprising molecular profiles of compositions having liver toxicities is therefore compiled. Those compositions causing cardiorvascular toxicities are similarly assessed for their molecular profiles and a library compiled. In addition, molecular profiles and library thereof for compositions having toxicities on central nervous system and for compositions having developmental toxicities are similarly established using the embryoid body system. The experimental procedures as described above in general, and in more detail in the following examples, are followed to compile the molecular profiles and libraries for compositions with particular type of toxicities.

Drugs with known or suspected of having activities against particular diseases can be used to establish molecular profiles and libraries for toxicity assessment. Antineuoplastics drugs with similar toxicities, for example those listed in Table 1, can be used to compile molecular profiles by determining the alterations in gene or protein expression patterns in embryoid bodies exposed to these drugs. Similarly, antibiotics with similar toxicities can also be assessed for their alterations in gene or protein expression patterns in embryoid bodies. Also used are drugs controlling diabetes, drugs for lowering lipid levels, or anti-inflammatory drugs. Once a composite library comprising molecular profiles of specific type of drugs having similar toxicities is established, it can be used to screen for new drug leads of the similar type for their potential toxicities. Again, the experimental procedures as described above in general, and in more detail in the following examples, are followed for compiling molecular profiles and libraries, and for typing/ranking toxicities of new drug leads.

### Example 2

### Establishing Protein Profiles for Chemical Agents Relating to Liver Toxicities

This Example demonstrates the culturing of embryoid bodies, the exposure of the embryoid bodies to different chemical agents having liver toxicities, and the determination of changes in protein expression in the embryoid bodies.

Five thousand CCE embryonic stem cells (Robertson, E., et al., Nature 323:445-448 (1986), were maintained and harvested according to Keller (Keller, G., et al., Mol. Cell Biol., 13:473-486 (1993). Briefly, the cells were cultured in 5 mls of IMDM medium, 20% FCS, ascorbic acid (50 μg/ml), and monothioglycerol (2.6×10⁻⁵ v/v) at 37° C. with 6% CO₂. On day 2, troglitazone, a drug marketed for the control of diabetes which has shown rare but severe liver toxicity, was added at a final concentration of 20 μM to one group of plates (group “A”) containing embryoid bodies. On that same day, erythromycin estolate (Sigma catalog E8630), a form of erythromycin with known liver toxicity, was added to a second group of plates (group “B”) at a final concentration of 50 μM. A third group of plates containing embryoid bodies (group “C1”) was cultured without any added drugs to serve as a control. Additionally, plates containing only tissue culture medium (group “C2”) were cultured alongside of those containing embryoid bodies as a control for degradation of proteins in the culture medium. After six days, and again at nine days, the cultures were harvested, the cells washed twice with PBS, and lysed in PBS, 0.5% Triton X100 for 10 minutes on ice. The nuclei were pelleted, and the supernatant removed and stored at −80° C. until analysis. The nuclei were lysed in PBS with 0.2% SDS and dounce homogenized to shear the DNA. The insoluble material was pelleted and the nuclear lysates stored at −80° C. until analyzed. Cytoplasmic and nuclear lysates were also taken on day zero prior to exposure to any test chemical compositions to serve as additional controls.

The lysates and medium samples were diluted 3 fold in buffer containing 50 mM Tris-HCl at pH 8, and 0.4 M NaCl. Aliquoted samples of diluted lysate or medium were placed in a sizing spin column that fractionated the sample with a 30 kD cutoff and equilibrated in 50 mM Tris-HCl, pH 8 and 50 mM NaCl. The column was spun at 700 g for 3 minutes for each fraction. Four fractions of 25 μL were collected for each column using the column equilibrated buffer.

The samples were partitioned by surface enhanced laser desorption/ionization (“SELDI”), and proteins were detected by mass spectroscopy. SELDI permits proteins to be captured on a surface of choice, which can then be washed at selected stringency, to permit fractionation according to desired characteristics such as affinity for metal ions of the surface used for capture.

Ciphergen normal phase chips (Ciphergen Biosystems, Palo Alto, Calif.) were used to partition the proteins in the fractions generated by the spin columns. One μL aliquots of each fraction were deposited on a spot on the chip, and the sample was air dryed at room temperature for 5 minutes. A mixture of 0.5 μL of saturated sinapinic acid (“SPA”) in 50% acetonitrile with 0.5% trifluroacetic acid (“TFA”) was applied to each spot. The chip was again permitted to air dry for 5 minutes at room temperature, and a second aliquot of the SPA mixture was applied.

Chips were read by the Ciphergen Protein Biology System 1 reader. Auto mode was used for data collection, at the SELDI quantitation setting. Two sets of protein profiles were collected, one at low laser intensity (at 15 with filter out) and one at high laser intensity (at 50 with filter out), detector set at 10. An average of 15 shots per location on the same sample spot were made. Protein profiles from different lysates were compared using SELDI software (Ciphergen Biosystems, Palo Alto, Calif.). This program assumes two proteins with a molecular weight within 1% of each other are the same. It then quantitates the results, compares the test samples against the control samples, and prints a graph showing the amount of each protein in the control as a horizontal line, with any reduction or the excess in the amount of each protein in the test sample compared to the amount of that protein in the control sample as a line below or above the line representing the control.

The results of these analyses for the day 6 embryoid bodies are shown as FIGS. 1 through 4. One portion of the results of this analysis, the differences in nuclear proteins expressed by the embryoid bodies, is shown in FIG. 1. The top panel, panel 1A, is a half-tone reproduction of the readout from the mass spectrometer. Viewing the sheet from along the long axis, the top band, is the mass spectrum for the control, the embryoid bodies grown in the absence of either of the test chemical compositions, the middle band is the spectrum for the embryoid bodies grown in the presence of added troglitazone, and the bottom band of FIG. 1A shows the mass spectrum of nuclear proteins expressed by embryoid bodies exposed to erythromycin estolate.

FIGS. 1B and 1C graphically depict differences in protein expression level between embryoid bodies contacted with one of the test chemical compositions (“test embryoid bodies”) and control embryoid bodies grown in standard tissue growth medium without added chemical compositions. These panels present computational subtractions of identical proteins between the respective test embryoid bodies and the control embryoid bodies to indicate only those proteins which are significantly different in expression between the test and the control embryoid bodies. Each bar represents a single protein and the length of the bar represents the amount of protein expressed by the embryoid bodies exposed to the test composition compared to the amount expressed by the control embryoid bodies. A bar above the center line indicates that the test embryoid body expressed more of that protein than did the control embryoid bodies; a bar below the line indicates that the test embryoid body expressed less of that protein.

FIG. 1B shows the differences in the nuclear proteins expressed by embryoid bodies grown in the presence of troglitazone compared to control embryoid bodies. FIG. 1C shows the differences in the nuclear proteins expressed by the embryoid bodies grown in the presence of erythromycin estolate and the control. (Both the test and the control embryoid bodies were at day 6 of development.) Reading FIGS. 1B and 1C from the left, the first bar encountered is above the line at the same position for both Figures, but the height of the bar is much greater in FIG. 1C. This indicates that both groups of test embryoid bodies expressed more of this protein than did the control, but that the bodies contacted with erythromycin estolate expressed considerably more than did bodies contacted with troglitazone.

Continuing along the X, or molecular weight, axis of FIG. 1C, the next four bars encountered also have a counterpart in FIG. 1B. Moreover, in each of the Figures, the bars representing the same three proteins are below the line, whereas the bar for the same fourth protein is above the line. Once again, the height of the lines differs between FIGS. 1C and 1B. Thus, for the first 5 nuclear proteins detected, the embryoid bodies contacted with troglitazone and with erythromycin estolate displayed the same pattern of protein expression, but at different levels of expression. Each of these proteins, and the overall expression pattern, would be a candidate for inclusion in a profile indicating that an unknown chemical composition, such as a new potential therapeutic, had some liver toxicity. Conversely, the first protein detected in FIG. 1C to the right of the 4000 Daltons molecular weight line does not have a counterpart (or at least a counterpart in terms of being expressed at a level different from that of the control bodies) in FIG. 1B. This protein would therefore not be considered a protein that demonstrated a common pathway of liver toxicity of both troglitazone and erythromycin estolate. Depending on its correlation with expression pathways of other hepatic toxins, it might, however, be associated with liver toxicity. Similar analyses can be made for the other proteins depicted on the two graphs.

A further way to present an analysis of the differences in protein expression can be seen in FIG. 2. FIG. 2 compares also the expression of small nuclear proteins in the three embryoid body groups described above. In these graphs, each bar in a panel represents a single protein, but the length of the bar represents the relative amount of protein expressed, rather than a comparison of the amount expressed compared to the control embryoid bodies. In FIG. 2, the top panel, 2A, graphs the level of protein expression, as determined by mass spectroscopy, in the embryoid bodies not exposed to chemical compositions in addition to those in a standard tissue culture medium. The middle panel, 2B, shows the level of expression of proteins of embryoid bodies exposed to troglitazone. And the bottom panel, 2C, shows the level of expression of embryoid bodies contacted with erythromycin estolate. In these panels, the expression level of the protein, plotted on the Y axis as a relative value, is plotted against the molecular weight, plotted on the X axis. A visual comparison of the panels reveals that some of the proteins expressed by the embryoid bodies exposed to the two drugs tested are the same, although perhaps at different levels of expression, and that others are different, and that both show a different pattern of expression than do the control embryoid bodies not exposed to either drug.

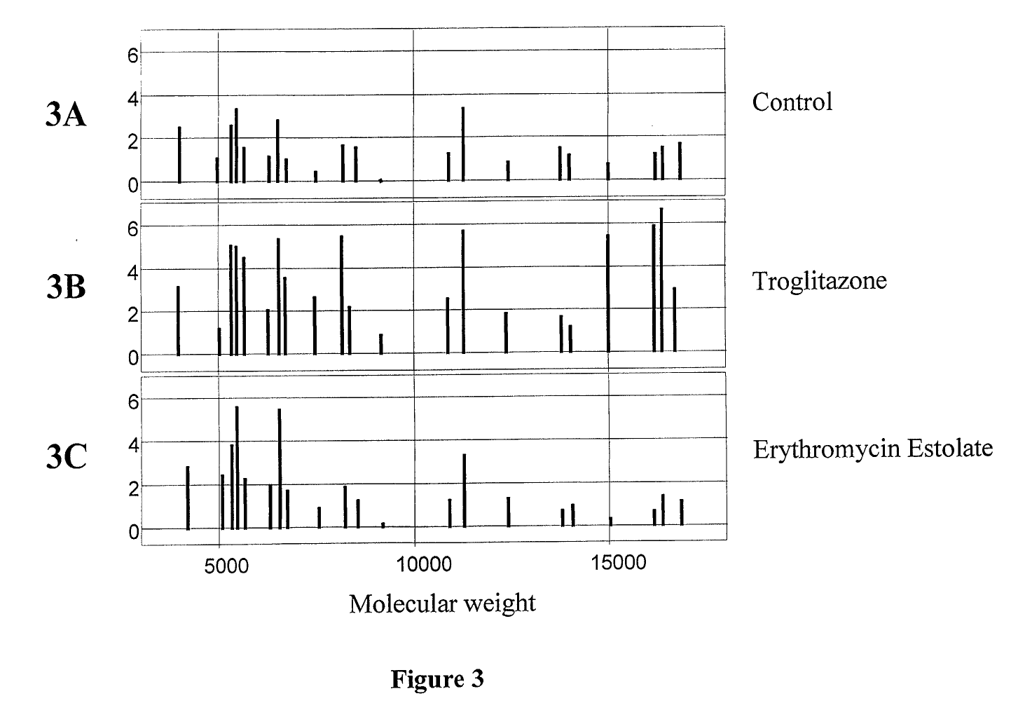

FIG. 3 shows the level of expression of small cytoplasmic proteins in the same three groups of embryoid bodies as those discussed in the preceding paragraph. The panels are arranged in the same order as in FIG. 2. Once again, the expression level of the protein for each group, plotted on the Y axis is plotted against the molecular weight of the proteins, plotted on the X axis. Once again, a visual comparison of the panels reveals that some of the proteins expressed by the embryoid bodies exposed to the two drugs tested are the same, although perhaps at different levels of expression, and that others are different.

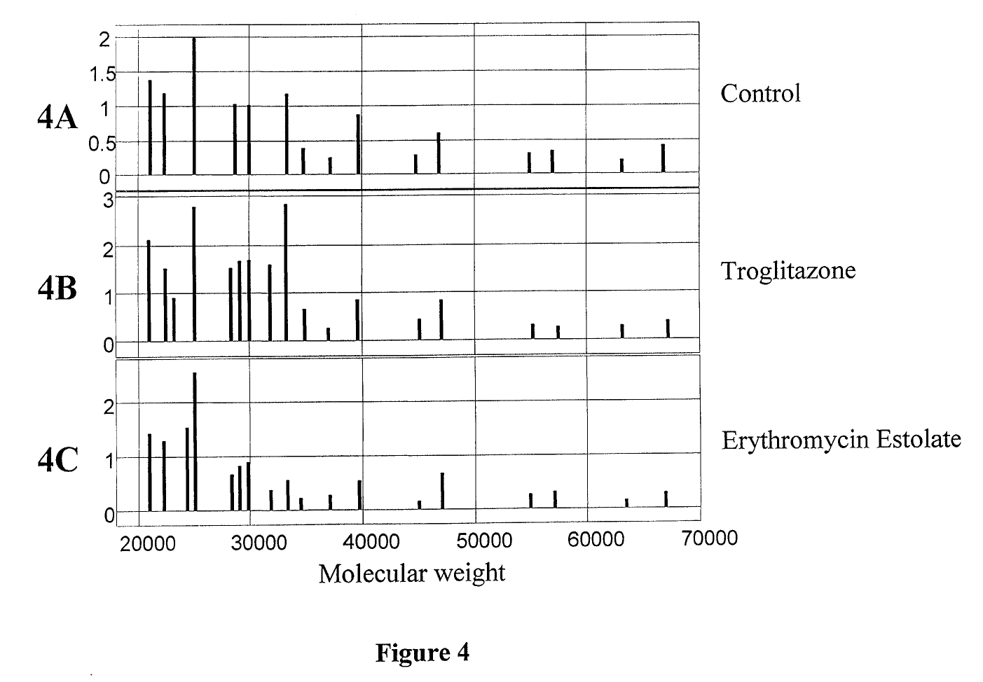

Similarly, FIG. 4 sets forth a graphical analysis of the large cytoplasmic proteins expressed by the same groups of embryoid bodies discussed above. Once again, the level of expression determined by the mass spectrometry is plotted on the Y axis, while the molecular weight is plotted on the X axis. Once again, clear similarities, and clear differences, can be observed between the protein expression patterns of the embryoid bodies exposed to the test chemical compositions, and between those protein expression patterns and that of the embryoid bodies grown without exposure to either of the test chemical compositions.

It is clear from these figures that the two drugs induce complex and unique protein expression patterns. Some proteins are expressed in smaller amounts (or “down regulated”) compared to the protein expression in the control embryoid bodies, and others are expressed in higher amounts (or “up regulated”) compared to the controls. Additionally, these two chemical compositions affect some of the same proteins and thus share common sub-patterns.

For example, in FIG. 2C, to the right of the line denoting a molecular weight of 2500 Daltons, there is a tall line, over 15 units on the Y axis, designating a strongly expressed protein. Following the line up to panels 2B and 2A, one can see that that same protein is expressed at high levels in both the embryoid bodies contacted with troglitazone and in the control embryoid bodies not contacted with either drug. This protein, therefore, is highly expressed in embryoid bodies at the point in development at which the samples were taken, although there is some variation in level of expression. Continuing to the right in panel 2C and making the same comparisons, however, the next protein present is also present, in approximately the same amount, in the embryoid bodies exposed to troglitazone, but is not expressed at all by the control embryoid bodies. Thus, this protein is a candidate for differentiating chemical compositions with liver toxicity from other compositions and other kinds of toxicity.

### Example 3

### Screening of Anti-cancer Drugs for Tissue and Organ Toxicities

This example illustrates using the EMBRYOID BODY system for screening anti-cancer agents for their tissue or organ toxicities.

Compounds and drugs (both anti-cancer and therapeutic) that have known toxicities and biology endpoints in humans and/or animals are selected for compiling their gene or protein expression profiles in embryoid bodies. In addition, compounds are selected with related known mechanisms of activities and with regard to compounds that have been used in previous studies to correlate clinical outcomes with human in vitro cell culture effects. Table 2.

TABLE 2

| | TOXICITIES | | | | | | | |
|---|---|---|---|---|---|---|---|---|
| DRUGS | DEV | LIVER | CV | GI | CNS | RENAL | BLOOD | MECHANISM |
| chloroquinoxaline | | | + | | | + | | ? |
| sulfonamide | | | | | | | | |
| didemnin B | | | | + | | | | ? |
| cyclosophosphamide | | | + | | | | | alkylator |
| bizelesin | | | | | | | + | alkylator |
| carboplatin | | | | + | | | + | alkylator |
| cisplatin | | | | + | | + | + | alkylator |
| oxaliplatin | | | | | + | | | alkylator |
| ectemascidin 743 | | | | | | | + | alkylator |
| penclomedine | | | | | + | | | alkylator |
| methotrexate | + | + | | | | | | anti-metabolite |
| fuzarabine | | | | | | | + | anti-metabolite |
| fludarabine | | | | | | | + | anti-metabolite |
| flavopiridol | | | | + | | | | CdK inhibitor |
| doxorubicin | | | + | | | | | DNA intercalator |
| amonafide | | | | | | | + | DNA intercalator |
| daunorubicin | | | + | | | | + | DNA syn inliib |
| gemcitabine | | + | | | | | + | DNA syn inhib |
| etoposide | | | | | | | + | DNA syn inhib |
| deoxyspergualin | | | + | | | | | immunosuppression |
| camptothecin | | | | | | | + | topo-I inhibitor |
| 9 aminocamptothecin | | | | | | | + | topo-I inhibitor |
| topotecan | | | | | | | + | topo-I inhibitor |
| merbarone | | | | | | + | | topo-Il inhibitor |
| dolastatin 10 | | | | | | | + | tubulin inhibitor |
| taxol | | | | | | | + | tubulin inhibitor |
| vinblastine | + | | | | | | + | tubulin inhibitor |
| vincristine | + | | | | | | + | tubulin inhibitor |
| vindesine | + | | | + | | | + | tubulin inhibitor |
| vinorelbine | + | | | | | | + | tubulin inhibitor |

“Dev” = developmental
“GI” = gastro-intestinal
“CV” =cardiovascular
“CNS” central nervous system

a. Establishing Gene Expression Profiles

The gene expression pattern of a selected compound is measured and quantified using cDNA microarrays and is normalized with cellular differentiation. The gene expression pattern of the compound is compared with a control EB culture not exposed to the compound or, where appropriate, EB cultures treated with related drugs with similar function or dose limiting toxicity. By compiling the gene expression profiles for a number of anti-cancer agents having similar or related toxicities, common alterations in gene expression are discerned and correlated with the toxicities, and are used as surrogate profiles for assessing the toxicities of test anti-cancer drug candidates.

The cDNA microarray can be any one of many kinds that are known and available in the art, for example, as described in Shalon et al (1996), Genome Res 6:639-645. cDNA microarrays allow for the simultaneous monitoring of the expression of thousands of genes, by direct comparison of control and chemically-treated cells. 3′ expressed sequence tags (ESTs) are arrayed and spotted onto glass microscope slides at a density of hundreds to thousands per slide using high speed robotics. Fluorescent CDNA probes are generated from control and test RNAs using a reverse transcriptase reaction with labeled dUTP using fluors that excite at two different wavelengths, i.e. Cy3 and Cy5, which allows for the hybridization of both the control and test RNA to the same chip for direct comparison of relative gene expression in each sample. The fluorescent signal is detected using a specially engineered scanning confocal microscope. A collection of 15,000 sequence verified human clones and 8700 mouse clones can be used in making cDNA microarrays. These microarrays are ideal for the analysis of gene expression patterns in EB cultures treated with a variety of agents.

Briefly, RNAs are isolated from control and treated EB cells. Total RNA are prepared using the RNAeasy kit from Qiagen. Subsequently, RNA are labeled either with Cy3 or Cy5 dUTP in a single round of reverse transcription. The resultant labeled cDNAs are mixed in a concentrated volume and hybridized to the arrays. Hybridizations is incubated overnight at 65° C. in a custom designed chamber that prevents evaporation. Following hybridization, the chip is scanned with a custom confocal laser scanner that will provide an output of the intensity of each spot in the array for both the Cy3 and Cy5 channels. The data is then analyzed with a software package that contains additional extensions. These extensions allow for the integration of a signal across each spot, normalization of the data to a panel of designated housekeeping genes, and statistical calculations to generate a list of genes whose ratios are outliers, or significantly changed by the treatment. In addition to the image analysis software, informatics packages such as Spot-Fire and GeneSpring, both are commercially available, are used to allow clustering and analysis of genes in multiple experiments across dose and/or time. CDNA microarray technology, in general, is still being validated as a viable technique for providing quantitative data. While the ratio of red/green provides good qualitative data on the relative level of expression of a gene in one population versus the other, it is not an absolute value of the level of induction/down regulation of that gene. Each pair of samples on the arrays are hybridized in triplicate. Outliers that are consistently induced or suppressed in two of the three hybridization experiments are further validated by a traditional RNA quantitation method, such as Northern blot or RT-PCR.

Each drug is tested at least three times on separate EB cultures for its effects on growth, differentiation and RNA expression. Cell counts (growth), colony counts (differentiation) and RNA levels (cDNA microarrays) are averaged for the three of more experiments and the mean and SEM determined. All results are normalized using approximately 15 “house keeping” genes. This allows a quantitative comparison of the effects of the test drugs to control compounds that are not toxic in humans or animals. Statistical comparisons provide information for determining whether a given drug affects EB cells gene expression compared to control drugs or non-treated cells and for determining whether a change in RNA in the cells is relevant.

b. Establishing Protein Expression Profiles

The protein expression profiles of the selected anti-cancer drugs are established using Ciphergen's SELDI mass spectroscopy (MS)-TOF system, as described in Example 2. Total cell lysates from harvested EB cultures are prepared in either 0.1% SDS or Triton-X 100 (0.5%) and directly applied to protein array chips using manufacture's protocols. Each chip can analyze two drugs in triplicate. After working out the stringency conditions and experimental replications, on average 6 ProteinChips™ per test compound are used.

The Ciphergen technology allows for the proteins in the sample to be captured, retained and purified directly on the chip. The proteins on the microchip are then analyzed by (SELDI). This analysis determines the molecular weight of proteins in the sample. An automatic readout of the molecular weights of the purified proteins in the sample can then be assessed. Typically this system has a CV of less than 20%. The Ciphergen data analysis system normalizes the data to internal reference standards and subtracts the readout of proteins found in control cells from those in drug treated cells. This data analysis reveals protein expression stimulated by the drugs as well as proteins only found in the control cells whose expression is inhibited by the drug. The analysis provides a qualitative readout of protein expression between a control and treated group. Analysis of multiple samples provides an average fold change in protein expression and a relative measure of variability. This can be represented as a mean±SEM which can provide a statistical measure of the protein changes. This analysis is used to determine whether drugs that induce similar forms of toxicity in humans cause similar changes in protein expression in EB cells. Each drug is analyzed on at least 3 separate groups of ES cells.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A library of molecular profiles of chemical compositions having predetermined toxicities, produced by a method comprising the steps of:
a) contacting an isolated mammalian embryoid body with a chemical composition having predetermined toxicities;
b) recording alterations in gene expression or protein expression in the mammalian embryoid body in response to the chemical composition to create a molecular profile of the chemical composition; and
c) compiling a library of molecular profiles by repeating steps a) and b) with at least two chemical compositions having predetermined toxicities.

2. The library of claim 1, wherein the isolated mammalian embryoid bodies are of human.

3. The library of claim 2, further wherein the chemical compositions having predetermined toxicities are selected from the group consisting of therapeutic agents, neurotoxins, renal toxins, hepatic toxins, toxins of hematopoietic cells, and myotoxins.

4. The library of claim 2, further wherein the chemical compositions having predetermined toxicities are selected from the group consisting of agents that are toxic to cells of one or more reproductive organs, teratogenic agents and carcinogens.

5. The library of claim 2, further wherein the chemical compositions having predetermined toxicities are selected from the group consisting of agricultural chemicals, cosmetics, and environmental contaminants.

6. The library of claim 1, wherein the isolated mammalian embryoid bodies are of non-human mammals.

7. The library of claim 6, wherein the non-human mammals are rodents.

8. The library of claim 6, further wherein the chemical compositions having predetermined toxicities are selected from the group consisting of animal therapeutics, neurotoxins, renal toxins, hepatic toxins, toxins of hematopoietic cells, and myotoxins.

9. The library of claim 6, further wherein the chemical compositions having predetermined toxicities are selected from the group consisting of agents that are toxic to cells of one or more reproductive organs, teratogenic agents and carcinogens.

10. The library of claim 6, further wherein the chemical compositions having predetermined toxicities are selected from the group consisting of agricultural chemicals, cosmetics, and environmental contaminants.

11. The library of claim 1, wherein the library comprises molecular profiles for at least 20 chemical compositions.
